(19) United States
(12) Patent Application Publication
(10) Pub. No.: US 2025/0302739 A1
(43) Pub. Date: Oct. 2, 2025

(54) LONG-ACTING INJECTABLE PHARMACEUTICAL COMPOSITIONS COMPRISING BIODEGRADABLE POLYMERS FOR DRUG DELIVERY

(71) Applicant: Foresee Pharmaceuticals Co., Ltd., Taipei City (TW)

(72) Inventors: Yuhua LI, Newark, DE (US); Shih-Fan JANG, New Taipei City (TW); Andrew Guarino, Newark, DE (US)

(73) Assignee: Foresee Pharmaceuticals Co., Ltd., Taipei City (TW)

(21) Appl. No.: 19/237,181

(22) Filed: Jun. 13, 2025

(51) Int. Cl. A61K 9/00; A61K 31/4745; A61K 31/495; A61K 47/20; A61K 47/34

(52) U.S. Cl. CPC A61K 9/0024; A61K 31/4745; A61K 31/495; A61K 47/20; A61K 47/34

(57) ABSTRACT

The present application relates to a sustained release delivery composition of a vesicular monoamine transporter type 2 (VMAT2) inhibitor, a deuterated derivative thereof, a pharmaceutically acceptable salt thereof, an active metabolite thereof, or a prodrug thereof for treatment of hyperkinetic movement disorders including, but not limited to, tardive dyskinesia (TD), Huntington's disease (HD) chorea, tremors, dystonia, chorea, tics, myoclonus, stereotypies, restless legs syndrome, and various other disorders with abnormal involuntary movements. The present application also relates to a sustained release delivery composition of an antipsychotic agent, a pharmaceutically acceptable salt thereof, an active metabolite thereof, or a prodrug thereof for treatment of schizophrenia, bipolar disorder, and other psychiatric diseases or disorders. The method of making or using the composition is also disclosed.

### 1. FIELD OF THE APPLICATION

The present application provides for a biodegradable polymeric depot composition which is stable and effective as a sustained release delivery system for biologically active compounds, specifically antipsychotic drugs and reversible human vesicular monoamine transporter type 2 (VMAT2) inhibitors. The composition of the present application comprises a) a VMAT2 inhibitor, including but not limited to, (3R, 11bR)-tetrabenazine [(+)-TBZ, (3R,11bR)-1,3,4,6,7,11b-hexahydro-9,10-dimethoxy-3-(2-methylpropyl)-2H-benzo[a]quinolizin-2-one], (2R,3R,11bR)-dihydrotetrabenazine [(+)-(α)-HTBZ, (2R,3R,11bR)-3-isobutyl-9,10-dimethoxy-1,3,4,6,7,11b-hexahydro-2H-pyrido[2,1-a]isoquinolin-2-ol)], (2S,3R,11bR)-dihydrotetrabenazine [(+)-(β)-HTBZ, (2S,3R,11bR)-3-isobutyl-9,10-dimethoxy-1,3,4,6,7,11b-hexahydro-2H-pyrido[2,1-a]isoquinolin-2-ol)], valbenazine [(2R,3R,11bR)-3-Isobutyl-9,10-dimethoxy-1,3,4,6,7,11b-hexahydro-2H-pyrido[2,1-a]isoquinolin-2-yl L-valinate], a deuterated derivative thereof, a pharmaceutically acceptable salt thereof, and a prodrug thereof; or an antipsychotic agent, included but not limited to cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT (a fixed dose combination of Xanomeline/trospium chloride), a pharmaceutically acceptable salt thereof, an active metabolite thereof, or a prodrug thereof; b) one or more biodegradable polymers, or copolymers, or block, or branched, or dendritic copolymers, or a mixture thereof; c) one or more pharmaceutically acceptable solvents, or a mixture thereof; and/or d) one or more optional pharmaceutically acceptable excipients to achieve optimal drug delivery. The present application also provides a method of manufacturing and the use in treating psychiatric diseases and disorders, such as schizophrenia and bipolar syndrome, and hyperkinetic diseases and disorders, such as tardive dyskinesia, by administration of such composition to human or a warm-blooded animal in need thereof.

### 2. DESCRIPTION OF THE RELATED ART

Tardive dyskinesia (TD) is a hyperkinetic movement disorder resulting in involuntary, repetitive body movements which are not related to other disorders provoking the aforementioned involuntary movements, for example, Parkinson's disease or tic disorders. Instead, TD is a neurological disorder most commonly caused by long-term use of dopamine blocking agents such as antipsychotic drugs (also known as neuroleptics or dopamine receptor antagonists). First generation neuroleptics (typical neuroleptics, for example haloperidol and chlorpromazine) are very likely to cause TD; while newer neuroleptics (atypical neuroleptics, for example aripiprazole and paliperidone), on the other hand, can do the same but to a lesser extent.

Prior arts suggest continuous exposure to neuroleptics can cause upregulation/supersensitiveness of dopamine receptor, which then induces hyperkinetic movement disorder. Vesicular monoamine transporter-2 (VMAT2) is a membrane protein that transports monoamine, such as dopamine, from presynaptic into synaptic vesicles. Therefore, many hyperkinetic movement disorders, namely TD, Tourette syndrome, and Huntington's disease can be reduced through depleting presynaptic dopamine by VMAT2 inhibitors. Tetrabenazine (TBZ, brand name XENAZINE®), known as cis-rac-1,3,4,6,7,11b-hexahydro-9,10-dimethoxy-3-(2-methylpropyl)-2H-benzo[a]quinolizin-2-one, is a potent and reversible inhibitor for human VMAT2 Ki˜ 100 nM (XENAZINE® Drug Approval Package, NDA 021894). However, while TBZ is orally administered as racemic mixtures, it is rapidly metabolized (majorly in the liver by carbonyl reductase) into four stereoisomeric metabolites: 2R,3R,11bR-HTBZ ((+)-α), 2S,3R,11bR-HTBZ ((+)-β), 2S,3S,11bS-HTBZ ((−)-α), and 2R,3S,11bS-HTBZ ((−)-β) (HTBZ, dihydrotetrabenazine, 9,10-dimethoxy-3-(2-methylpropyl)-2,3,4,6,7,11b-hexahydro-1H-benzo[a]quinolizin-2-ol) (Skor H. et el., Drugs R D. 2017 September; 17(3):449-459). However, each metabolite shows varied affinity to rat VMAT2: Ki is 4.2, 9.7, 250, and 690 nM, respectively corresponding to 2R,3R,11bR-HTBZ ((+)-α), 2S,3R,11bR-HTBZ ((+)-β), 2S,3S,11bS-HTBZ ((−)-α), and 2R,3S,11bS-HTBZ ((−)-β) (Grigoriadis et al., Journal of Pharmacology and Experimental Therapeutics June 2017, 361 (3) 454-461).

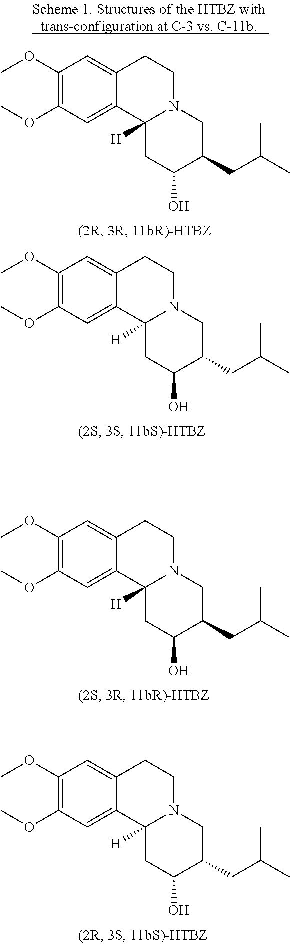

In addition, 2S,3S,11bS-HTBZ ((−)-α) and 2R,3S,11bS-HTBZ ((−)-β) have high off-target binding affinity to dopamine D2 and serotonin 5-HT₇ receptors (180/71 nM and 53/5.9 nM for ((−)-α) and ((−)-β), respectively), which results in severe side effects of TBZ administration (i.e., insomnia, tremor, rigid muscle, problems with balance etc.) (Harriott et al., Progress in Medicinal Chemistry Volume 57, 2018, Pages 87-111). Moreover, due to the variable CYP2D6-mediated metabolism of TBZ, the maintenance dose of TBZ varies from one individual to another, CYP2D6 inducers or inhibitors should also be avoided for subjects taking TBZ. What's even more significant and potentially inconvenient is that metabolism variation between patients makes dose titration unavoidable for conventionally available TBZ medications. Furthermore, the side effects related to TBZ such as sedation, depression, akathisia and Parkinsonism and therapeutic variability have impeded its application potential.

In 2017, two new medications were approved to treat TD: Valbenazine (VBZ) (INGREZZA®, Neurocrine Biosciences, Inc., single 40 mg or 80 mg capsule per day) and deutetrabenazine (AUSTEDO®, Teva, 6 mg, 9 mg, or 12 mg tablet, twice daily). Unlike TBZ, deutetrabenazine and VBZ have pharmacokinetic advantages which enable less frequent dosing for better tolerability. VBZ, L-Valine, (2R,3R,11bR)-1,3,4,6,7,11b-hexahydro-9,10-dimethoxy-3-(2-methylpropyl)-2H-benzo[a]quinolizin-2-yl ester, is an ester of (+)-(α)-HTBZ with L-valine. By solely introducing (+)-(α)-HTBZ without the presence of the other side effect inducing stereoisomeric metabolites, such as (−)-(α)-HTBZ and (−)-(β)-HTBZ, VBZ is considered much more tolerable and safer than TBZ and dose titration is not needed. On the other hand, in the case of AUSTEDO®, deuterated derivative of TBZ increases its half-life which benefits for reduced dosing frequency (thrice daily vs twice daily).

Although the success of INGREZZA® and AUSTEDO® improve TD treatment in oral dosage forms, both products still require daily dosing, which can result in lack of patient adherence.

More than 150 million people have been diagnosed with schizophrenia and bipolar syndrome, which are chronic conditions. These people are at risk for substance abuse and suicide. The symptoms of these diseases can be controlled and stabilized with proper medication and adherence to the dosing regimens. Likewise, Vraylar®, Latuda®, and Rexulti®, common antipsychotics used to treat schizophrenia and bipolar disorder, also require daily oral dosing. Poor compliance remains the most critical challenge in treatment of chronic illness. Schizophrenia, for example, is often associated with cognitive dysfunction, lack of motivation, depression, and demoralization. While the introduction of antipsychotics can be backdated to the 1950s, poor patient adherence to oral dosage forms has always been a crucial issue. Relapse is a continual risk in schizophrenia patients and represents one of the major public health problems associated with such illness. The use of long-acting injectables (LAIs) can alleviate the burden of frequent administration which helps improve patient medication adherence.

Approved LAIs for schizophrenia, such as Risperdal Consta®, requires multiple reconstitution steps prior to intramuscular (IM) injection. Other long-acting antipsychotics on the market, such as Abilify®, Invega Sustena®, Invega Trinza®, and Invega Hafyera® consist of drug particles as powders or aqueous suspensions. While aqueous media is commonly used in injectables, it has potential drawbacks, such as chemical instability through hydrolytic reactions, particle aggregation, crystal growth, particle sedimentation due to low viscosity, and potential for microbial growth.

Therefore, there is an unmet medical need for a stable, safer, and user-friendly LAI medication for the treatment of involuntary movement disorders with significantly reduced dosing frequency and improved patient compliance. There is also a need for LAI formulations of antipsychotic drugs to better support patient compliance and comfort, while making it easier for health care administrators to prepare and administer as well.

### 3. SUMMARY OF THE APPLICATION

The present application provides polymer depot compositions comprising of a) a VMAT2 inhibitor, including but not limited to, tetrabenazine (TBZ), (3R,11bR)-tetrabenazine [(+)-TBZ, (3R,11bR)-1,3,4,6,7,11b-hexahydro-9,10-dimethoxy-3-(2-methylpropyl)-2H-benzo[a]quinolizin-2-one], (2R,3R,11bR)-dihydrotetrabenazine [(+)-(α)-HTBZ, (2R,3R,11bR)-3-isobutyl-9,10-dimethoxy-1,3,4,6,7,11b-hexahydro-2H-pyrido[2,1-a]isoquinolin-2-ol)], (2S,3R,11bR)-dihydrotetrabenazine [(+)-(β)-HTBZ, (2S,3R,11bR)-3-isobutyl-9,10-dimethoxy-1,3,4,6,7,11b-hexahydro-2H-pyrido[2,1-a]isoquinolin-2-ol)], valbenazine [(2R,3R,11bR)-3-Isobutyl-9,10-dimethoxy-1,3,4,6,7,11b-hexahydro-2H-pyrido[2,1-a]isoquinolin-2-yl L-valinate], a deuterated derivative thereof, a pharmaceutically acceptable salt thereof; or an antipsychotic agent, included but not limited to cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a pharmaceutically acceptable salt thereof, an active metabolite thereof, and a prodrug thereof, b) one or more biodegradable polymers, or copolymers, or block, or branched, or dendritic copolymers, or a mixture thereof; c) one or more pharmaceutically acceptable solvents, or a mixture thereof; and d) one or more optional pharmaceutically acceptable excipients to achieve optimal drug delivery for intended uses.

The present application relates to a long-acting injectable delivery system of (+)-TBZ, (+)-(α)-HTBZ, (+)-(β)-HTBZ, valbenazine, a deuterated derivative thereof, a pharmaceutically acceptable salt thereof, an active metabolite thereof, or a prodrug thereof, which have high VMAT2 receptor binding affinity (<10 nM), but low off-target binding to such as dopamine, serotonin, and adrenergic receptors (>1000 nM). The present application also relates to a long-acting injectable delivery system of cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a pharmaceutically acceptable salt thereof, an active metabolite thereof, or a prodrug thereof, for treating a psychiatric disease or disorder in a subject.

Appropriately, the present application provides a stable, biodegradable composition that is effective as an in situ forming depot allowing prolonged, controlled release of (+)-TBZ, (+)-(α)-HTBZ, (+)-(β)-HTBZ, valbenazine, cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT (a fixed dose combination of Xanomeline/trospium chloride), a deuterated derivative thereof, a pharmaceutically acceptable salt thereof, an active metabolite thereof, or a prodrug thereof. The present polymer depot compositions can be a viscous fluid, a solution, a gel, an emulsion, a suspension, or a semisolid dispersion that is preserved in a readily pre-filled syringe for subcutaneous (SC) or intramuscular (IM) injection. The polymer depot compositions can also be stabilized and preserved in two separated containers such as vials, ampules, cartridges, or syringes, i.e., one container contains the active pharmaceutical ingredient and the other one contains the delivery vehicle. After adequately mixing the two containers together, the final mixture can be a viscous fluid, a solution, a gel, an emulsion, a suspension, or a semisolid dispersion for subcutaneous or intramuscular injection.

Specifically, the present application is capable of forming a sustained release implant/depot upon administration to a living subject at the injection site. Preferably, the inventive compositions are competent for maintaining long-term plasma concentration of (+)-TBZ, (+)-(α)-HTBZ, (+)-(β)-HTBZ, valbenazine, cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, or related active metabolites above the therapeutic level, preferably for 1 to 2 weeks, more preferably for 2 to 4 weeks, and most preferably for 1 to 3 months with minimum variation on plasma concentration and narrow peak-to-trough (P/T) ratio, so as to limit potential off-target effects (for example resulted from the (−)-stereoisomers of TBZ and HTBZ) and ultimately provide an improved safety profile to solve the unmet medical need of currently available drug products on the market.

### 4. BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1. In vitro Release of Sustained-Release Depot Pharmaceutical Composition Composed of (+)-(α)-HTBZ and Poly(lactide)-PEG Diblock Copolymer.

FIG. 2. In vitro Release of Sustained-Release Depot Pharmaceutical Composition Composed of (+)-(α)-HTBZ-Poly(lactic-co-glycolic acid)-Polyethylene glycol Triblock Copolymer and (+)-(α)-HTBZ-Poly(lactide)-PEG-Poly (lactic-co-glycolic acid)-Polyethylene glycol Copolymers (A Combinations of both Diblock and Triblock Copolymers).

### 5. DETAILED DESCRIPTION OF THE APPLICATION

It must be noted that as used herein and in the appended claims, the singular forms “a,” “an,” and “the” and similar referents are to be construed to cover both the singular and the plural, unless otherwise indicated herein or clearly contradicted by context.

As used herein, in the context of the present application, all numbers disclosed herein are approximations, whether or not the words “about” or “approximately” are used. Each numerical number means a range of the numerical value ±10% of the numerical value unless otherwise indicated. For example, “about 100 mL” or “100 mL” includes any values between 90 and 110 mL.

As used herein, the term “about” or “approximately” preceding a numerical value or a series of numerical values means ±10% of the numerical value unless otherwise indicated. For example, “approximately 100 mg” means 90 to 110 mg.

Unless otherwise indicated, the term “at least” preceding a series of elements is to be understood to refer to every element in the series. Those skilled in the art will recognize or be able to ascertain using no more than routine experimentation, many equivalents to the specific embodiments of the application described herein. Such equivalents are intended to be encompassed by the application.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “subject” as used herein refers to any individual or patient to which the subject methods are performed. Generally, the subject is human, although as will be appreciated by those in the art, the subject may be an animal. The terms “subject” and “patient” are used interchangeably. In some embodiments, the subject is a mammal. In some embodiments, the subject is a human. In some embodiments, the subject is an animal, such as a mouse, rat, rabbit, dog, monkey, or a laboratory test animal, etc.

The present application relates to a polymeric, biodegradable, biocompatible long-acting injectable drug delivery system suitable for in-situ formation of a depot or an implant to deliver pharmaceutically active ingredients in a controlled and sustained manner. The preferred polymer depot composition of the present application is a combination of a) a VMAT2 inhibitor, including but not limited to, (3R,11bR)-tetrabenazine [(+)-TBZ, (3R,11bR)-1,3,4,6,7,11b-hexahydro-9,10-dimethoxy-3-(2-methylpropyl)-2H-benzo[a]quinolizin-2-one], (2R,3R,11bR)-dihydrotetrabenazine [(+)-(α)-HTBZ, (2R,3R,11bR)-3-isobutyl-9,10-dimethoxy-1,3,4,6,7,11b-hexahydro-2H-pyrido[2,1-a]isoquinolin-2-ol)], (2S,3R,11bR)-dihydrotetrabenazine [(+)-(β)-HTBZ, (2S,3R,11bR)-3-isobutyl-9,10-dimethoxy-1,3,4,6,7,11b-hexahydro-2H-pyrido[2,1-a]isoquinolin-2-ol)], valbenazine, or an antipsychotic agent, including but not limited to cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a deuterated derivative thereof, a pharmaceutically acceptable salt thereof, an active metabolite thereof, or a prodrug thereof; b) one or more biodegradable polymers, or copolymers, or block, or branched, or dendritic copolymers, or a mixture thereof; c) one or more pharmaceutically acceptable solvents, or a mixture thereof; and d) one or more optional pharmaceutically acceptable excipients to achieve optimal drug delivery for the intended use.

As used herein, the term of TBZ is defined as tetrabenazine, (+)-TBZ or 1,3,4,6,7,11b-hexahydro-9,10-dimethoxy-3-(2-methoxylrpopryl)-2H-benzo(a)quinoline-2-one). It is a reversible inhibitor of vesicular monoamine transporter 2 (VMAT-2).

As used herein, the term of (+)-TBZ is defined as (+)-tetrabenazine, (3R,11bR)-TBZ, or (3R,11bR)-tetrabenazine.

As used herein, the term of (−)-TBZ is defined as (−)-tetrabenazine, (3R,11bS)-TBZ, or (3R,11bS)-tetrabenazine.

As used herein, the term of VBZ is defined as valbenazine or L-Valine, (2R,3R,11bR)-1,3,4,6,7,11b-hexahydro-9,10-dimethoxy-3-(2-methylpropyl)-2H-benzo[a]quinolizin-2-yl ester.

As used herein, the term of (±)-d6-TBZ is defined as deutetrabenazine, or racemic deutetrabenazine. Deutetrabenazine is a hexahydro-dimethoxybenzoquinolizine derivative and has the following chemical name: (RR, SS)-1,3,4,6,7,11b-hexahydro-9,10-di(methoxy-d3)-3-(2-methylpropyl)-2H-benzo[a]quinolizin-2-one. Deutetrabenazine is a racemic mixture containing RR-deutetrabenazine ((+)-d6-TBZ) and SS-deutetrabenazine ((−)-d6-TBZ).

As used herein, the term of (+)-d6-TBZ) is defined as RR-deutetrabenazine and the term of (−)-d6-TBZ is defined as SS-deutetrabenazine.

As used herein, the term of (+)-(α)-HTBZ is defined as (+)-α-dihydrotetrabenazine, one of the metabolites of tetrabenazine.

As used herein, the term of (+)-(□)-HTBZ is defined as (+)-□-dihydrotetrabenazine, one of the metabolites of tetrabenazine.

As used herein, the term of (−)-(α)-HTBZ is defined as (−)-α-dihydrotetrabenazine, one of the metabolites of tetrabenazine.

As used herein, the term of (−)-(□)-HTBZ is defined as (−)-□-dihydrotetrabenazine, one of the metabolites of tetrabenazine.

As used herein, the term of (+)-d6-(α)-HTBZ is defined as (+)-d6-alpha-dihydrotetrabenazine, one of the metabolites of deutetrabenazine.

As used herein, the term of (−)-d6-(α)-HTBZ is defined as (−)-d6-alpha-dihydrotetrabenazine, one of the metabolites of deutetrabenazine.

As used herein, the term of (+)-d6-(β)-HTBZ is defined as (+)-d6-beta-dihydrotetrabenazine, one of the metabolites of deutetrabenazine.

As used herein, the term of (−)-d6-(β)-HTBZ is defined as (−)-d6-beta-dihydrotetrabenazine, one of the metabolites of deutetrabenazine.

As used herein, the term “antipsychotic agent” refers to any substance that lessens the symptoms of a psychotic disorder. Such examples include, but are not limited to, free base or pharmaceutically accepted salts or esters of cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, or related active metabolites.

As used herein, the term “psychotic disorder” refers to a disorder in which psychosis is a recognized symptom, which includes neuropsychiatric and neurodevelopment disorders, neurodegenerative disorders, depression, mania, schizophrenic disorders, and bipolar disorders. Preferably, this relates to schizophrenic and bipolar disorders.

The present polymer depot compositions can be a viscous fluid, a solution, a gel, an emulsion, a suspension, or a semisolid dispersion that is preserved in a pre-filled syringe and ready for subcutaneous or intramuscular injection.

The polymer depot compositions can also be stabilized and preserved in two separated syringes. In one syringe (A), dry powders of (+)-TBZ, (+)-(α)-HTBZ, (+)-(β)-HTBZ, valbenazine, cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a deuterated derivative thereof, a pharmaceutically acceptable salt thereof, an active metabolite thereof, or a prodrug thereof, is pre-filled, while the other syringe (B) is filled with a delivery vehicle that is comprised of one or more biodegradable, biocompatible polymers, one or more biocompatible organic solvent(s) and optional pharmaceutically acceptable excipient(s). Prior to injection, syringe A and syringe B are connected via a connector, followed by mixing the components thoroughly in turns of pushing the two plungers back-and-forth for a sufficient number of times. Preferably, syringe A is a male luer lock syringe, while syringe B is a female luer lock syringe that can be connected directly to each other and can be disconnected from each other easily. More preferably, syringe A and syringe B are polymer prefillable syringes that are suitable for terminal sterilization, including but not limited, to E-beam, X-ray, and gamma-irradiation. The final mixed formulation ready for injection can be a viscous liquid, a solution, a gel, an emulsion, a suspension, or a semisolid dispersion, which is stable preferably within about 30 minutes and more preferably within about 1-2 hours post-mixing.

The polymer depot compositions in this invention can be administrated via aforementioned syringes or devices thereof to a living subject subcutaneously, intramuscularly, intraperitoneally, or intradermally and form a depot or an implant in-situ at the injection site. As soon as the polymer depot composition comes in contact with an aqueous medium or body fluid, the biocompatible organic solvent(s) dissipates from the polymer depot composition, leaving the biodegradable, biocompatible, polymeric carrier to form a depot, or to precipitate and form a solid matrix which encapsulates the pharmaceutically active ingredients including but not limited to TBZ, (+)-TBZ, (+)-(α)-HTBZ, (+)-(α)-HTBZ, valbenazine, cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a deuterated derivative thereof, a pharmaceutically acceptable salt thereof, an active metabolite thereof, or a prodrug thereof.

As used herein, the term “VMAT2” is the abbreviation of vesicular monoamine transport type 2. VMAT2 inhibitors are agents that cause a depletion of neuroactive peptides, such as dopamine in nerve terminals and are used to treat chorea due to neurodegenerative diseases (such as Huntington's disease) or dyskinesia due to neuroleptic medications (tardive dyskinesia, TD). As of 2022, three VMAT2 inhibitor drug products have become available in the United States to manage dyskinesia syndromes, each with a somewhat different spectrum of approved indications: tetrabenazine (XENAZINE® and generics: 2008), deutetrabenazine (AUSTEDO®: 2017) and valbenazine (INGREZZA®: 2017). VMAT2 inhibitors have not been associated with serum enzyme elevations during therapy or linked to instances of clinically apparent liver injury, but they have had limited general clinical use.

As used herein, a VMAT2 inhibitor includes, but is not limited to, tetrabenazine (TBZ), dihydrotetrabenazine (HTBZ), deutetrabenazine (d6-TBZ), and deuterated dihydrotetrabenazine (d6-HTBZ), (3R,11bR)-tetrabenazine [(+)-TBZ, (3R,11bR)-1,3,4,6,7,11b-hexahydro-9,10-dimethoxy-3-(2-methylpropyl)-2H-benzo[a]quinolizin-2-one], (2R,3R,11bR)-dihydrotetrabenazine [(+)-(α)-HTBZ, (2R,3R,11bR)-3-isobutyl-9,10-dimethoxy-1,3,4,6,7,11b-hexahydro-2H-pyrido[2,1-a]isoquinolin-2-ol)], (2S,3R,11bR)-dihydrotetrabenazine [(+)-(□)-HTBZ, (2S,3R,11bR)-3-isobutyl-9,10-dimethoxy-1,3,4,6,7,11b-hexahydro-2H-pyrido[2,1-a]isoquinolin-2-ol)], valbenazine, a deuterated derivative thereof, a pharmaceutically acceptable salt thereof, an active metabolite thereof, or a prodrug thereof.

Tetrabenazine, a hexahydro-dimethoxy-benzoquinolizine derivative, acts primarily as a reversible high-affinity inhibitor of mono-amine uptake into granular vesicles of presynaptic neurons by binding selectively to VMAT2. [Kenney C, Jankovic J. Tetrabenazine in the treatment of hyperkinetic movement disorders. Exp Rev Neurother. 2006; 6(1):7-17]. Both tetrabenazine (TBZ) and its active metabolite dihydrotetrabenazines (HTBZ) are potent inhibitors of VMAT2.

Tetrabenazine is rapidly and extensively metabolized by first-pass metabolic reduction of the 2-keto group, generating four stereoisomeric metabolites namely (2R,3R,11bR)-HTBZ, (2S,3S,11bS)-HTBZ, (2S,3R,11bR)-HTBZ, and (2R,3S,11bS)-HTBZ. The four TBZ metabolites are likely the major pharmacologically active substances in vivo. The primary pharmacological action of TBZ and its active metabolites is to deplete the levels of monoamines (e.g., dopamine, serotonin, and norepinephrine) within the central nervous system by inhibiting human VMAT2 [D. Scherman, B. Gasnier, P. Jaudon, J. P. Henry, Mol. Pharmacol. 33 (1988) 72-77; A. Pletscher, A. Brossi, K. F. Gey, Int. Rev. Neurobiol. 4 (1962) 275-306; A. P. Vartak, J. R. Nickell, J. Chagkutip, L. P. Dwoskin, P. A. Crooks, J. Med. Chem. 52 (2009) 7878-7882]. This transporter is predominantly expressed in human brain, which translocates monoamines from cytoplasm into synaptic vesicles, where they are both stored and protected from metabolism prior to their synaptic release. Multiple lines of evidence indicate that the binding of TBZ and its metabolites to VMAT2 is stereospecific [M. Kilbourn, L. Lee, T. V. Borght, D. M. Jewett, K. Frey, Eur. J. Pharmacol. 278 (1995) 249e252; M. R. Kilbourn, L. C. Lee, M. J. Heeg, D. M. Jewett, Chirality 9 (1997) 59e62; M. R. Kilbourn, L. C. Lee, D. M. Jewett, R. A. Koeppe, K. A. Frey, J. Cereb. Blood Flow Metab. 15 (1995) S650]. Tetrabenazine enantiomers and all eight stereoisomers of dihydrotetrabenazine were synthesized and evaluated as VMAT2 inhibitors [Zhangyu Yao, Xueying Wei, Xiaoming Wu, Jonathan L. Katz, Theresa Kopajtic, Nigel H. Greig, and Hongbin Sun, European Journal of Medicinal Chemistry 46 (2011) 1841-1848]. Among the TBZ enantiomers and eight HTBZ isomers, (+)-TBZ, (+)-(α)-HTBZ and (+)-(β)-HTBZ demonstrated relatively high rat VMAT2 binding affinity of 4.47, 3.96, and 13.4 nM, respectively.

As used herein, the VMAT2 inhibitor is (3R,11bR)-tetrabenazine, or (3R,11bR)-1,3,4,6,7,11b-hexahydro-9,10-dimethoxy-3-(2-methylpropyl)-2H-benzo[a]quinolizin-2-one, or (+)-TBZ.

As used herein, the VMAT2 inhibitor is referred to (2R,3R,11bR)-9,10-dimethoxy-3-(2-methylpropyl)-2,3,4,6,7,11b-hexahydro-1H-benzo[a]quinolizin-2-ol, or (2R,3R,11bR)-dihydrotetrabenazine, or (+)-α-3-isobutyl-9,10-dimethoxy-1,3,4,6,7,11b-hexahydro-2H-pyrido[2,1-a]isoquinolin-2-ol, or (+)-alpha-dihydrotetrabenazine, or (+)-(α)-HTBZ, or (+)-(α)-DTBZ, or (+)-(α)-DHTBZ. These abbreviations are used interchangeably herein. “(+)-α-HTBZ” is one of the active metabolites of tetrabenazine.

As used herein, the VMAT2 inhibitor is (2S,3R,11bR)-1,3,4,6,7,11b-Hexahydro-9,10-dimethoxy-3-(2-methylpropyl)-2H-benzo[a]quinolizin-2-ol, or (2S, 3R,11bR)-dihydrotetrabenazine, or (+)-(β)-3-isobutyl-9,10-dimethoxy-1,3,4,6,7,11b-hexahydro-2H-pyrido[2,1-a]isoquinolin-2-ol, or (+)-beta-dihydrotetrabenazine, or (+)-(β)-HTBZ, or (+)-(β)-DTBZ, or (+)-(β)-DHTBZ. These abbreviations are used interchangeably herein. “(+)-(β)-HTBZ” is one of the active metabolites of tetrabenazine.

As used herein, deutetrabenazine is an isotopic isomer of tetrabenazine in which six hydrogen atoms have been replaced by deuterium atoms. The incorporation of deuterium slows down drug metabolism, prolongs drug half-life, therefore, allowing less frequent dosing [Coppen EM, Roos RA, “Current Pharmacological Approaches to Reduce Chorea in Huntington's Disease”. Drugs. 77 (2017): 29-46]. Deutetrabenazine is extensively metabolized by the liver into active metabolites including deuterated alpha-dihydrotetrabenazine (alpha-HTBZ) and deuterated beta-dihydrotetrabenazine (beta-HTBZ).

Valbenazine [(2R,3R,11bR)-3-Isobutyl-9,10-dimethoxy-1,3,4,6,7,11b-hexahydro-2H-pyrido[2,1-a]isoquinolin-2-yl L-valinate], a benzoquinolizidine derivative, is first approved by the FDA in 2017 (INGREZZA®, Neurocrine Biosciences, 40 mg and 80 mg capsules) for the treatment of TD in adults. VBZ is formulated as a tosylated salt which is slight water solubility. VBZ and its pharmaceutically active metabolite, (+)-(α)-HTBZ, are potent VMAT2 inhibitors with Ki of about 150 nM and about 3 nM, respectively, with no off-targeting binding affinity to dopaminergic, serotonergic, or adrenergic receptors (Ki>5000 nM). VBZ is primarily metabolized via hydrolysis of the valine ester bond, followed by metabolism via cytochrome P450 3A4/5 into (+)-(α)-HTBZ [Stepan Uhlyar and Jose A. Rey. “Valbenazine (Ingrezza)”, Pharmacy & Therapeutics, June 2018; 43(6): 328-331].

The preferred VMAT2 inhibitor has low off-target binding affinity. More preferably, the VMAT2 inhibitor is (+)-TBZ, (+)-(α)-HTBZ, (+)-(β)-HTBZ, valbenazine, a deuterated derivative thereof, a pharmaceutically acceptable salt thereof, an active metabolite thereof, or a prodrug thereof. The deuterated derivatives include deuterated TBZ, deuterated (+)-TBZ, deuterated (+)-(α)-HTBZ, deuterated (+)-(β)-HTBZ, and the like.

In a preferred embodiment, the VMAT2 inhibitor is (+)-TBZ. (+)-TBZ is optically purified from racemic TBZ where the other stereoisomer (−)-TBZ is removed. Racemic TBZ can be rapidly metabolized to its four reduced form (+)-(α)-HTBZ, (−)-(α)-HTBZ, (+)-(β)-HTBZ and (−)-(β)-HTBZ in vivo. Among those, (−)-(α)-HTBZ and (−)-(β)-HTBZ are likely to be responsible for the cause of serious side effects due to high alterative binding to dopamine D2s and serotonin 5-HT receptors. In this particular embodiment, using optically pure (+)-TBZ as the only pharmaceutically active ingredient would significantly lower the risk of severe side effects generated from off-target binding, which provides a much preferred and safer drug product.

In another preferred embodiment, VMAT2 inhibitor is (+)-(α)-HTBZ or (+)-(β)-HTBZ. Both (+)-(α)-HTBZ and (+)-(β)-HTBZ are the reduced forms of (+)-TBZ. (+)-(α)-HTBZ and (+)-(β)-HTBZ can be generated from (+)-TBZ in vivo majorly in the liver by carbonyl reductase or, can also be easily synthesized by a person of ordinary skill in the art. Instead of the parent compound, a single active metabolite can further guarantee minimal metabolism variation between patients (especially for patients with CYP 2D6 polymorphism) that can generate additional complications while receiving VMAT2 inhibitors.

The polymer depot composition of the present application is produced by combining a VMAT2 inhibitor including (+)-TBZ, (+)-(α)-HTBZ, (+)-(β)-HTBZ, valbenazine, or an antipsychotic agent including cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a deuterated derivative thereof, a pharmaceutically acceptable salt thereof, an active metabolite thereof, or a prodrug thereof with a solution of a solid, biodegradable, biocompatible polymer dissolved in one or more pharmaceutically acceptable and biocompatible solvents. The polymer depot composition can be administered by a syringe and a needle to a patient in need of treatment. Any suitable biodegradable polymer can be employed, provided that the biodegradable polymer is at least substantially insoluble in body fluid.

The application is based in part on the discovery that incorporation of a VMAT2 inhibitor or an antipsychotic agent in a viscous depot vehicle produces a formulation that has low initial burst release, minimal lag time, and near zero-order release in vivo. For a depot formulation, this release profile is surprising because other evidence in prior arts disclosed that a low burst, near zero-order release is virtually impossible to achieve unless special steps being taken, such as coating and (micro) encapsulation.

The polymer depot composition according to embodiments of the application can be prepared as injectables. The administration route may include a subcutaneous, intramuscular, intramyocardial, adventitial, intratumoral, or intracerebral. Multiple or repeated injections may be administered to a subject to maintain a therapeutic effect or to a subject that requires further administration of the drug for any reason. The polymer depot composition serves as an implanted sustained release drug delivery system after injection into the subject. Such controlled release can be over a period of one week, more than one week, one month, or more than one month. Preferably, the controlled release is over at least a period of one week, more preferably over a period of at least one month.

In certain embodiments of the application, the vehicle of viscous depot includes a biocompatible copolymer and/or co-oligomer, i.e., a polymer that would not cause irritation or necrosis in the tissues of the subjects. The biocompatible copolymers of the application may be bioerodible, i.e., gradually decompose, dissolve, hydrolyze and/or erode in situ. Examples of bioerodible copolymers and/or co-oligomer include, but are not limited to, polylactides, polyglycolides, polycaprolactones, polyanhydrides, polyamines, polyurethanes, polyesteramides, polyorthoesters, polydioxanones, polyacetals, polyketals, polycarbonates, polyorthocarbonates, polyphosphazenes, poly (malic acid), poly (amino acids), polyvinylpyrrolidone, polyethylene glycol, polyhydroxycellulose, polysaccharides, chitin, chitosan, and mixtures thereof. The copolymer and/or co-oligomer can be in linear, di-block, tri-block, branched or dendritic structure. The copolymer and/or co-oligomer is dissolved in a pharmaceutically acceptable solvent and is typically present in the solution in an amount ranging from about 5 to about 80% by weight, preferably from about 20 to 70%, often more preferably from about 30 to about 65% by weight.

In one embodiment, the copolymer is a block copolymer. The block copolymer is composed of one or more hydrophilic block(s) linked with one or more hydrophobic block(s). A hydrophilic block is composed of, but not limited to, poly (malic acid), poly (amino acids), polyvinylpyrrolidone, polyethylene glycol (PEG), or the combination thereof. A hydrophobic block is composed of, but not limited to polylactides (or polylactic acid, PLA), poly(lactide-coglycolide) or poly(lactic-co-glycolic acid) (PLGA or PLG), polycaprolactones (PCL), polyanhydrides, polyurethanes, polyesteramides, polyorthoesters, polydioxanones, polyacetals, polyketals, polycarbonates, polyorthocarbonates, polyphosphazenes, or the combination thereof.

PLGA or PLG is a copolymer which is used in a host of Food and Drug Administration (FDA) approved therapeutic devices, owing to its biodegradability and biocompatibility. PLGA is synthesized by means of ring-opening co-polymerization of two different monomers, the cyclic dimers (1,4-dioxane-2,5-diones) of glycolic acid and lactic acid. Polymers can be synthesized as either random or block copolymers thereby imparting additional polymer properties.

A polylactide polymer is a polymer based on lactic acid. The term “lactic acid” as used herein includes the isomers L-lactic acid, D-lactic acid, DL-lactic acid, L-lactide, D-lactide, and DL-lactide. Polylactide, also known as poly (lactic acid) or polylactic acid (abbreviation PLA), is a thermoplastic polyester with backbone formula (C₃H₄O₂)n or [—C(CH₃)HC(═O)O-]n, formally obtained by condensation of lactic acid C(CH₃)(OH)HCOOH by removing water (H₂O). It can also be prepared by ring-opening polymerization of lactide [—C(CH₃)HC(═O)O—]₂, the cyclic dimer of the basic repeating unit. Polylactide contains an asymmetric a-carbon which is typically described as the D or L form in classical stereochemical terms and sometimes as R and S form, respectively. The enantiomeric forms of the polymer PLA are poly D-lactic acid (PDLA) and poly L-lactic acid (PLLA). The term “polylactide” as used herein includes poly(L-lactic acid), poly(D-lactic acid), poly(DL-lactic acid), poly(L-lactide), poly(D-lactide), and poly(DL-lactide).

In one embodiment, the biocompatible copolymer is a block copolymer. The biocompatible block copolymer is composed of one or more hydrophilic PEG block linked with one or more hydrophobic PLA block. The block copolymer can be a diblock, a triblock copolymer of PLA and PEG, or the combination thereof. The block copolymer of PLA-PEG used herein can be purchased from various suppliers such as Evonik and Ashland. In 2018, Evonik published “RESOMER® product brochure” including the “Resomer® Select naming” as shown below (https://healthcare.evonik.com/en/drugdelivery/parenteral-drug-delivery/parenteral-excipients/bioabsorbable-polymers/standard-polymers. Downloaded on Jan. 8, 2024).

### RESOMER® RP D Catalog of PLA-PEG Block Copolymers

The RESOMER® RP d catalog includes di-block copolymer: of poly(DL-lactide) and poly(ethylene glycol) incorporating various weight fractions of miPEG-5000. These amphiphilic polymers are available to help schleve shorter degradation times than standard lactsie/glycolide-containing polymers and are especially suited for nanoparticle formation.

POLY(DL-LACTIDE)-CO-POLY(ETHYLENE GLYCOL) RESOMER ® RP
D STANDARD PRODUCTS

| | Inherent | | PEG | Degradation |
|---|---|---|---|---|
| Polymer name | viscosity (dl/g) | Composition | (wt %) | timeframe |
| RESOMER ® | 0.55-0.68 | Poly(DL-lactide)-block-poly(ethylene | 15 | <2 months |
| RP d 155 | | glycol) methyl ether 5000, 15 wt. % | | |
| | | PEG | | |
| RESOMER ® | 0.39-0.49 | Poly(DL-lactide)-block-poly(ethylene | 25 | <2 months |
| RP d 255 | | glycol) methyl ether 5000, 25 wt. % | | |
| | | PEG | | |
| RESOMER ® | 0.33-0.42 | DL-lactide)-block-poly(ethylene glycol) | 33 | <2 months |
| RP d 335 | | methyl ether 5000, 33 wt. % PEG | | |

In some embodiments, the biocompatible copolymer is a di-block copolymer or a triblock copolymer or a combination of as disclosed in the U.S. Pat. Nos. 9,023,897, 11,666,527, 11,612,563, 10,646,443, 12,128,132, 11,801,217, 11,865,205, EP2866837B1, and U.S. Pat. No. 11,813,359 which are referenced in their entirety.

In some embodiments, the biodegradable copolymer is a di-block copolymer having the formula:

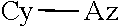

wherein A is a polyester selected from the group of, polylactic acid, polyglycolic acid, polycaprolactone, polyethylene adipate, polyhydroxyalkanoate and mixtures thereof and C is an end-capped polyethylene glycol, preferably methoxy polyethylene glycol and y and z are the number of repeat units with y ranging from 2 to 400 and z ranging from 1 to 3,000.

In a preferred embodiment, the biodegradable copolymer is a diblock copolymer having the formula:

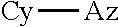

wherein A is polylactide and C is polyethylene glycol and y and z are the number of repeat units with y ranging from 2 to 50 and z ranging from 4 to 350.

In some embodiments, the biodegradable copolymer is a tri-block copolymer having the formula:

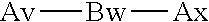

wherein A is a polyester selected from the group of, polylactic acid, polyglycolic acid, polycaprolactone, polyethylene adipate, polyhydroxyalkanoate and mixtures thereof and B is polyethylene glycol and v and x are the number of repeat monomer units ranging from 1 to 3,000 and w is the number of repeat monomer units ranging from 3 to 300 and v=x or v≠x.

In a preferred embodiment, the biodegradable copolymer is a triblock copolymer having the formula:

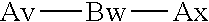

wherein A is polylactide and B is polyethylene glycol and v and x are the number of repeat monomer units ranging from 4 to 700 and w is the number of repeat monomer units ranging from 3 to 275 and v=x or v≠x.

In one embodiment the composition further comprises at least one organic solvent. Typically, the organic solvent is a pharmaceutically acceptable solvent. This organic solvent can be retained in the composition or evaporated off prior to administration.

In one embodiment the composition further comprises:
(a) a biodegradable triblock copolymer having the formula:

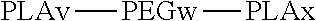

wherein v and x are the number of repeat units ranging from 1 to 3,000 and w is the number of repeat units ranging from 3 to 300 and v=x or v≠x; and/or
(b) a biodegradable diblock copolymer having the formula:

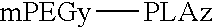

wherein y and z are the number of repeat units with y ranging from 2 to 400 and z ranging from 1 to 3,000.

In a preferred embodiment the composition comprises:
(a) a biodegradable triblock copolymer having the formula:

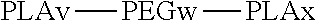

wherein v and x are the number of repeat units ranging from 4 to 700 and w is the number of repeat units ranging from 3 to 275 and v=x or v≠x; and/or
(b) a biodegradable diblock copolymer having the formula:

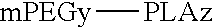

wherein y and z are the number of repeat units with y ranging from 2 to 50 and z ranging from 4 to 350, which are dissolved in a pharmaceutically acceptable solvent, such as DMSO or NMP.

In some embodiments the composition comprises:
(a) a biodegradable triblock copolymer having the formula:

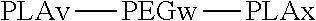

wherein v and x are the number of repeat units ranging from 1 to 3,000 and w is the number of repeat units ranging from 3 to 300 and v=x or v≠x; and
(b) 1, 2, 3 or 4 different biodegradable diblock copolymers each having the formula:

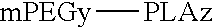

wherein y and z are the number of repeat units with y ranging from 2 to 400 and z ranging from 1 to 3,000;
and wherein the weight ratio between (a) and (b) is 1:19 to 5:1.

In some preferred embodiments the composition comprises:
(a) a biodegradable triblock copolymer having the formula:

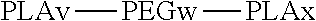

wherein v and x are the number of repeat units ranging from 4 to 700 and w is the number of repeat units ranging from 3 to 275 and v=x or v≠x; and
(b) 1, 2, 3 or 4 different biodegradable diblock copolymers each having the formula:

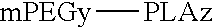

wherein y and z are the number of repeat units with y ranging from 2 to 50 and z ranging from 4 to 350;
and wherein the weight ratio between (a) and (b) is 1:10 to 2:1.

In some embodiments the composition comprises: (a) two different biodegradable triblock copolymers each having the formula:

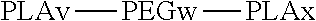

wherein v and x are the number of repeat units ranging from 1 to 3,000 and w is the number of repeat units ranging from 3 to 300 and v=x or v≠x; and
(b) 1, 2, 3 or 4 different biodegradable diblock copolymer(s) each having the formula:

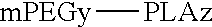

wherein y and z are the number of repeat units with y ranging from 2 to 400 and z ranging from 1 to 3,000;
and wherein the weight ratio between (a) and (b) is 1:19 to 5:1.

In some preferred embodiments the composition comprises: (a) two different biodegradable triblock copolymers each having the formula:

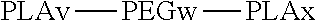

wherein v and x are the number of repeat units ranging from 4 to 700 and w is the number of repeat units ranging from 3 to 275 and v=x or v≠x; and
(b) 1, 2, 3 or 4 different biodegradable diblock copolymer(s) each having the formula:

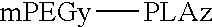

wherein y and z are the number of repeat units with y ranging from 2 to 50 and z ranging from 4 to 350;
and wherein the weight ratio between (a) and (b) is 1:10 to 2:1.

Typically, the composition is an injectable liquid and is suitable for forming a depot when injected into the body or are small solid particles or rod implants or spatial formulations.

Typically, the mass of the polyethylene glycol chain ranges from 180 g/mol to 12 kg/mol or 194 g/mol to 12 kg/mol or 200 g/mol to 12 kg/mol or from 100 g/mol to 4 kg/mol and the molecular weight of the end-capped polyethylene glycol chain ranges from 100 g/mol to 2 kg/mol or 164 g/mol to 10 kg/mol.

The biodegradable drug delivery composition may also further comprise a pharmaceutically acceptable vehicle, such as a solvent.

In one embodiment the pharmaceutically active ingredient is hydrophobic.

In one embodiment the pharmaceutically active ingredient is VMAT2 inhibitor, including but not limited to (±)-TBZ (TBZ racemate), (±)-(α)-HTBZ ((α)-HTBZ racemate), (±)-(b)-HTBZ ((b)-HTBZ racemate), (+)-TBZ, (+)-(α)-HTBZ, (+)-(b)-HTBZ, valbenazine, a deuterated derivative thereof, a pharmaceutically acceptable salt thereof, an active metabolite thereof, or a prodrug thereof.

In another embodiment the pharmaceutically active ingredient is an antipsychotic agent, including but not limited to cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a pharmaceutically acceptable salt thereof, an active metabolite thereof, or a prodrug thereof.

In another embodiment the at least one pharmaceutically active ingredient is present in an amount of from 0.05% to 60% (w/w %), optionally 0.05% to 40%, optionally 0.05% to 30%, optionally 0.05% to 10%, optionally 0.05% to 7%, optionally 0.05% to 2% of the total composition.

In one embodiment the biodegradable drug delivery composition is an injectable liquid and the at least one pharmaceutically active ingredient is present in an amount of 0.05% to 60% (w/w %). The injectable liquid can be in the form of a solution or emulsion. In the solution, the pharmaceutically active ingredient is completely dissolved in the pharmaceutically acceptable solvent along with the biodegradable polymer to form a clear solution. In the case of an emulsion, the pharmaceutically active ingredient is dissolved in the pharmaceutically acceptable solvent and suspended as solvent-API rich liquid droplets within the biodegradable polymer-solvent solution.

In another embodiment the biodegradable drug delivery composition is an injectable suspension and the at least one pharmaceutically active ingredient is present in an amount of 0.05% to 60% (w/w %). The injectable suspension is a viscous drug product where the pharmaceutically active ingredient is suspended as solid particles in the biodegradable drug delivery vehicle.

In an alternative embodiment the biodegradable drug delivery composition is a rod implant and the at least one pharmaceutically active ingredient is present in an amount of from 50% to 80% (w/w %).

In a further embodiment, the copolymers are present in an amount of 2% to 60% (w/w %) of the total composition, optionally 10% to 50%, optionally 20% to 40%, optionally 20% to 35%, optionally 30% to 50%.

In one embodiment the one or more triblock copolymers are present in an amount of 1% to 50% (w/w %), optionally 5% to 40% of the total composition.

In one embodiment the one or more di-block copolymers are present in an amount of 1% to 57% (w/w %), 2.5% to 45% of the total composition.

In an additional embodiment the weight ratio of the sum of the biodegradable triblock copolymers of (a) over the sum of the biodegradable di-block copolymers of (b) in said biodegradable drug delivery composition is 1:5 to 3:1.

Typically, the polyester repeat unit to ethylene oxide molar ratio in the composition is between 0.5 to 22.3, optionally 0.5 to 10, optionally 0.5 to 3.5 in the triblock and 0.8 to 15, optionally 1 to 10 in the di-block.

The composition may comprise three different block copolymers as defined above or four different block copolymers as defined above or five different block copolymers as defined above or six different block copolymers as defined above.

The composition may comprise one biodegradable triblock copolymer as defined above, or two different biodegradable triblock copolymers as defined above, or three different biodegradable triblock copolymers as defined above, or four different biodegradable triblock copolymers as defined above.

The composition may comprise one biodegradable di-block copolymer as defined above, or two different biodegradable di-block copolymers as defined above, or three different biodegradable di-block copolymers as defined above, or four different biodegradable di-block copolymers as defined above.

In one embodiment the composition comprises a triblock copolymer present in an amount of 1% to 50% (w/w %) of the total composition, a di-block copolymer presents in an amount of 1% to 57% (w/w %) of the total composition, and one or more further di-block or triblock copolymers each present in an amount of 0.5 to 20 (w/w %) of the total composition.

Typically, the release of at least one active ingredient is modulated by the composition. The release of VMAT2 inhibitors or antipsychotic agents from the biodegradable drug delivery composition of the present invention, can be released gradually over an extended period of time. This slow release can be continuous or discontinuous, linear or nonlinear and can vary due to the composition of the triblock copolymer and diblock copolymer. The higher the lactic acid content of the triblock and diblock copolymers, as well as chain lengths of the triblock and diblock copolymers present in the biodegradable drug composition, the longer the duration of the release. In another embodiment, the release profile could be fine-tuned by a combination of poly(lactide)-PEG diblock copolymer and poly (lactic-co-glycolic acid)-polyethylene glycol triblock copolymer. For example, a higher ratio of the poly(lactide)-PEG diblock copolymer in a pharmaceutical composition could accelerate the release rate due to the more hydrophilic nature of the poly(lactide)-PEG diblock copolymer, compared to the more hydrophobic poly(lactic-co-glycolic acid)-polyethylene glycol triblock copolymer. Such an unexpected finding could be advantageous for fine tuning the release. Additionally, this means that a lower overall polymer content could be used to slow down the release, by adjusting the ratio of triblock to diblock copolymers and reducing the overall diblock copolymer content. Having less polymer and a higher solvent content would result in a more fluid formulation and allow for use of a smaller needle for the injection.

In one embodiment the composition is suitable to deliver the active ingredient, optionally a therapeutically effective amount of the active ingredient, to a subject for at least 1 day, optionally at least 3 days, optionally at least 7 days, optionally at least 30 days, optionally at least 90 days, optionally at least 1 year.

In one embodiment the composition is suitable for parenteral administration.

In one embodiment the block copolymers are substantially insoluble in an aqueous solution, optionally wherein the block copolymers have less than 5%, optionally less than 1% (w/w) solubility in an aqueous solution.

In a further aspect, the present invention provides a method of modulating the kinetics of release of at least one active ingredient, said method comprising administering a biodegradable drug delivery composition as defined in any preceding claim to a subject, wherein the release kinetics of said at least one active ingredient from said biodegradable drug delivery composition are modulated without affecting one or more physical parameters of said biodegradable drug composition.

Typically, the one or more physical parameters are injectability and viscosity before injection of the biodegradable drug delivery composition and depot robustness after injection of the biodegradable drug delivery composition.

In one embodiment, the biocompatible copolymer is a branch copolymer. The biocompatible branch copolymer is composed of one or more hydrophilic branch linked with one or more hydrophobic branch. The hydrophilic branch is composed of, but not limited to poly(malic acid), poly(amino acids), polyvinylpyrrolidone, and polyethylene glycol (PEG), polyglycolide [poly(glycolic acid), PGA] or the combination thereof. The hydrophobic branch is composed of, but not limited to polylactides (or polylactic acid, PLA), polycaprolactones (PCL), polyanhydrides, polyurethanes, polyesteramides, polyorthoesters, polydioxanones, polyacetals, polyketals, polycarbonates, polyorthocarbonates, polyphosphazenes, or the combination thereof.

In another embodiment, the biocompatible copolymer is a dendritic (or so-called star polymer) copolymer composed of glucose and branched poly(DL-lactide-co-glycolide) arms. The dendritic glucose-PLGA polymers used herein can be purchased from various suppliers such as Evonik and Ashland. In 2018, Evonik published “RESOMER® product brochure” including the “Resomer® Select naming” as shown below (https://healthcare.evonik.com/en/drugdelivery/parenteral-drug-delivery/parenteral-excipients/bioabsorbable-polymers/standard-polymers, downloaded on Jan. 8, 2024).

GLUCOSE STAR POLYMER

| | | | Inherent | |
|---|---|---|---|---|
| Product | | | viscosity | End |
| no. | Polyraer name | Abbrev | (IV) dL/g | group |
| B6133-1 | 55:45 Poly(DL-lactide- | 55:45 DL- | 0.40-0.60** | Hydroxyl |
| | co-glycolide), Glucose | PLG-Glu | | |
| | initiated | | | |

**value measured in chloroform

PLGA or PLA degrades by hydrolysis of its ester linkages in the presence of water. It has been shown that the time required for degradation of PLGA is related to the monomers' ratio in the PLGA: the higher the content of glycolide units, the shorter the time required for degradation, as compared to predominantly lactide materials, PLA. In addition, polymers that are end-capped with esters (as opposed to free carboxylic acid terminated) demonstrate longer degradation half-lives [Samadi, N.; Abbadessa, A.; Di Stefano, A.; van Nostrum, C. F.; Vermonden, T.; Rahimian, S.; Teunissen, E. A.; van Steenbergen, M. J.; Amidi, M. & Hennink, W. E. (2013). “The effect of lauryl capping group on protein release and degradation of poly(D,L-lactic-co-glycolic acid) particles”. Journal of Controlled Release. 172 (2): 436-443]. This flexibility in degradation has made it convenient for fabrication of many medical devices, such as, grafts, sutures, implants, prosthetic devices, surgical sealant films, micro and nanoparticles [Pavot, V; Berthet, M; Rességuier, J; Legaz, S; Handké, N; Gilbert, SC; Paul, S; Verrier, B (December 2014). “Poly(lactic acid) and poly(lactic-co-glycolic acid) particles as versatile carrier platforms for vaccine delivery”. Nanomedicine (Lond.). 9 (17): 2703-18]. In a block copolymer-based sustained-release drug delivery system, the release pattern and duration of the LAI depot can be tailored through molecular weight (Mw) of the copolymers comprising of, ratio of the diblock and triblock, composition of the overall copolymer, as well as content % of the copolymer comprising of [Feifei Ng, Victor Nicoulin, Charlotte Peloso, Silvio Curia, Joël Richard, and Adolfo Lopez-Noriega (November 2023). “In Vitro and In Vivo Hydrolytic Degradation Behaviors of a Drug-Delivery System Based on the Blend of PEG and PLA Copolymers”. ACS Appl. Mater. Interfaces 2023, 15, 48, 55495-55509].

In certain embodiments of the application, the block copolymer is practically insoluble in aqueous medium or in body fluid, yet is readily soluble or miscible in biocompatible organic solvents to form a solution, a viscous fluid, or a semisolid.

In still another embodiment, the desired biodegradable, biocompatible, polymeric carrier is, but not limited, to diblock or triblock copolymers of PLA and PEG. Both diblock and triblock copolymers are soluble in biocompatible solvents or a combination of biocompatible solvents. Once dissolved in such biocompatible solvents or a combination thereof, viscous delivery vehicles can be formed. The delivery vehicles can subsequently be formulated with one or more pharmaceutically active ingredients to form the polymeric drug delivery compositions of the application, where the one or more pharmaceutically active ingredients can either be completely dissolved or suspended in the polymeric drug delivery composition of the application. As soon as the polymeric drug delivery composition contacts with an aqueous medium or body fluid, the biocompatible organic solvents dissipate out, leaving the biodegradable, biocompatible polymer to form a gel or semisolid depot, or to precipitate and form a solid matrix which encapsulates the antipsychotic agent, such as cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a pharmaceutically acceptable salt thereof, an active metabolite thereof, or a prodrug thereof, or VMAT2 inhibitors, such as (+)-TBZ, (+)-(α)-HTBZ, (+)-(β)-HTBZ, valbenazine, a deuterated derivative thereof, a pharmaceutically acceptable salt thereof, an active metabolite thereof, or a prodrug thereof, which is then released in a controlled and sustained manner for a duration of at least one week and more preferably of a least one month.

The pharmaceutically acceptable and biocompatible solvents in the present application are water soluble, miscible to dispersible or at least showing partial solubility in water. As used herein, the terms “soluble” and “miscible” are meant to be used interchangeably. When combined with biodegradable, hydrophobic polymers, the solvents can readily solvate the said polymers, resulting in delivery vehicles with desired viscosity. The delivery vehicles can be further formulated with pharmaceutically active ingredients to form the polymer depot compositions of the application to achieve controlled and sustained drug delivery. Examples of the pharmaceutically acceptable and biocompatible solvents include, but are not limited to, ethanol (EtOH), 1-Methyl-2-pyrrolidone or N-methyl-2-pyrrolidone (NMP), benzyl benzoate (BB), benzyl alcohol (BA), dimethyl sulfoxide (DMSO), tetraglycol (or glycofurol), dimethylacetamide (DMAc), triacetin (TA), low molecular weight polyethylene glycol (i.e. PEG 300 and PEG 400), polyethylene glycol esters, methyl acetate, ethyl acetate, ethyl oleate, glycerol, esters of caprylic and/or capric acids with glycerol or alkylene glycols, and the combination thereof.

In one preferred embodiment, the pharmaceutically acceptable and biocompatible solvent is N-Methyl-2-pyrrolidone (NMP).

In another preferred embodiment, the pharmaceutically acceptable and biocompatible solvent is dimethyl sulfoxide (DMSO).

According to the present application, the polymer depot composition comprises at least one biodegradable, biocompatible polymer and at least one pharmaceutically acceptable solvent to form the delivery vehicle. Preferably, the biodegradable, biocompatible polymer is substantially water-insoluble, which precipitates or forms a water-insoluble depot or implant after injection. In a preferred embodiment, block copolymers composed of PLA and PEG as defined herein is used to prolong the release of VMAT2 inhibitors such as (+)-TBZ, (+)-(α)-HTBZ, (+)-(β)-HTBZ, valbenazine, a deuterated derivative thereof, a pharmaceutically acceptable salt thereof, an active metabolite thereof, or a prodrug thereof. The inventive compositions are preferably to maintain plasma concentrations of (+)-TBZ, (+)-(α)-HTBZ and (+)-(β)-HTBZ at or above therapeutic level preferably for 1 to 2 weeks, more preferably for 2 to 4 weeks, and most preferably for 1 to 3 months with minimum variation in plasma concentration and narrow peak to trough (P/T) ratio.

In another preferred embodiment, block copolymers composed of PLA and PEG as defined herein are used to prolong the release of antipsychotic agents such as cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a pharmaceutically acceptable salt thereof, an active metabolite thereof, or a prodrug thereof. The inventive compositions are preferably to maintain plasma concentrations of the antipsychotic agent at or above therapeutic level preferably for 1 to 2 weeks, more preferably for 2 to 4 weeks, and most preferably for 1 to 3 months with minimum variation in plasma concentration and narrow peak to trough (P/T) ratio.

In one embodiment, the VMAT2 inhibitors and antipsychotic agents are soluble in the biocompatible solvent and when mixed with the biodegradable polymer solution will be completely dissolved and form a liquid solution or emulsion, or gel. In another embodiment, the VMAT2 inhibitors and antipsychotic agents have limited to no solubility in the biocompatible solvent so that when mixed with the biodegradable polymer solution will remain as solid particles suspended in the viscous polymer solution.

Generally speaking, particle size can alter the release profile in suspension formulations (Drug Des. Devel. Ther. 2013; 7:1027-1033.). Dissolution rate is positively correlated to the surface area of the particles in a suspension formulation. While specific surface area increases with decreasing particle size of the drug, so does the drug dissolution rate. A substantial difference in dissolution rate can exist according to the variation on particle size and the relative surface area, especially during the initial period of the dissolution. In the present application, API particle size is tailored as an effective approach on tuning for desirable release profiles for VMAT2 inhibitors and antipsychotic agents.

The present application further provides methods of preparing and using such polymer depot compositions. In one embodiment, a method of preparing such compositions comprising of (+)-TBZ, (+)-(α)-HTBZ, (+)-(β)-HTBZ, valbenazine, a deuterated derivative thereof, a pharmaceutically acceptable salt thereof, an active metabolite thereof, or a prodrug thereof, one or more biocompatible organic solvents, and one or more pharmaceutically acceptable polymeric, water-insoluble carriers. Preferably, the pharmaceutically acceptable polymeric, water-insoluble carrier is dissolved, or mixed with at least one biocompatible organic solvent(s) to form the delivery vehicle first, followed by dissolving or suspending (+)-TBZ, (+)-(α)-HTBZ, (+)-(β)-HTBZ, valbenazine, a deuterated derivative thereof, a pharmaceutically acceptable salt thereof, an active metabolite thereof, or a prodrug thereof in the delivery vehicle.

In another embodiment, a method of preparing such compositions comprising of cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a deuterated derivative thereof, a pharmaceutically acceptable salt thereof, an active metabolite thereof, or a prodrug thereof, one or more biocompatible organic solvents, and one or more pharmaceutically acceptable polymeric, water-insoluble carriers. Preferably, the pharmaceutically acceptable polymeric, water-insoluble carrier is dissolved, or mixed with at least one biocompatible organic solvent(s) to form the delivery vehicle first, followed by dissolving or suspending cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a deuterated derivative thereof, a pharmaceutically acceptable salt thereof, an active metabolite thereof, or a prodrug thereof in the delivery vehicle.

The present inventive polymer depot composition can be a viscous fluid, semi-solid, emulsion, or uniform suspensions ready for injection in a pre-filled syringe. The preferred composition can also be a homogeneous, viscous fluid, semi-solid, or uniform suspensions after adequate mixing prior to injection. Such compositions are physio-chemically stable prior to and during the preparation process. Preferably, such compositions are stable during manufacturing, sterilization, storage, and subsequent administration to a living subject. The polymer depot composition is preferably to be administrated via syringes or similar devices thereof to a living subject subcutaneously, intramuscularly, intraperitoneally, or intradermally and form an in-situ forming depot or implant. Preferably, the polymer depot composition of the present application has an initial release in vivo no more than 30% within 24 hours, more preferably no more than 20% within 24 hours, most preferably, no more than 10% within 24 hours. With the desired components, the polymer depot composition can sustainably deliver the pharmaceutical active ingredient above the therapeutic level preferably for 1 to 2 weeks, more preferably for 2 to 4 weeks, and most preferably for 1 to 3 months with minimum variation in plasma concentration and narrow P/T ratio (preferably from 1-10 and more preferably from 1-4, and still more preferably from 1 to 2), which can surely help limit potential side effects so as to provide an improved safety profile for patients. The polymer depot compositions are biocompatible and degradable in a living subject, followed by body absorption once drug delivery is complete.

### 6. EXAMPLES

The following examples demonstrate the compositions and methods of the present application. The following examples should not be considered as limitations, but should merely teach those skill in the art how to make the effective sustained release injectable polymer depot compositions.

### Example 1. HPLC Analytical Method: VMAT2 Inhibitors

A calibration curve was obtained through the HPLC method below to quantify the concentration of TBZ, (+)-TBZ, (+)-HTBZ, valbenazine, and antipsychotic compounds in a sample with unknown API content.

### Materials

### Reagents

Mill-Q water, resistivity greater than 18.0 MΩ-cm, or equivalent.
Ammonium acetate, ACS grade or equivalent
Sodium hydroxide, ACS grade or equivalent
Acetonitrile (ACN), HPLC grade
Isopropyl alcohol (IPA), HPLC grade
N-Methyl-2-pyrrolidone (NMP), HPLC grade

### Reference Standards

(+)-TBZ API with the defined potency

### Instruments and Parameters

### HPLC

Shimadzu HPLC System:
Binary Pump: Model LC-20AT
Degasser: Model DGU-20A3R
Autosampler: Model SIL-30A HT
Column Oven: Model CTO-20AC
Detector: Model SPD-20A

### Parameters

Column: BEH C18 Column, 1.7 μm 3.0×150 mm
Mobile Phase A: 2 mM ammonium phosphate/Acetonitrile (ACN) 9/1;
Mobile Phase B: ACN/Mill-Q water 9/1
Isocratic mode: A/B=42/58
Flow rate: 0.7 mL/min
Column temp: 35° C.
Injection vol: 2 μL
Detection: 220 nm
Run time: 6.5 min

### Sample Preparation

### Mobile Phase A

Dissolve about 0.294 g of ammonium phosphate in 1000 mL water. Filter through 0.22 μm PTFE membrane filter and degas before use.

### Sample Solvent:

Isopropyl alcohol

### Standard Solution

Accurately weigh 20±1 mg of (+)-TBZ Reference Standard into a 20 mL volumetric flask, add 10 mL of sample solvent to dissolve it, dilute to the volume with sample solvent and mix well. Dilute this solution with sample solvent to obtain standard solutions at 2, 5, 10, 50, 100, 200, and 500 μg/mL.

### Sample Solution

For (+)-TBZ API samples (0.1 mg/ml of TBZ):
   Accurately weigh 10 mg of API sample into a 10 mL volumetric flask, add 5 mL of sample solvent to dissolve it, dilute to the volume with sample solvent and mix well. Pipette 1 mL of above solution into a 10 mL volumetric flask, dilute with sample solvent to volume, and mix well.
For (+)-TBZ drug product samples (0.1 mg/ml of TBZ):
   Accurately weigh 40 mg of drug product (assuming drug loading is 50%, w/w) sample into a 20 mL volumetric flask, add 15 mL of NMP to dissolve it, dilute to the volume with NMP and mix well. Take 1 mL of the sample solution prepared above, added into a 10 mL volumetric flask, add 5 mL of IPA to dilute it, dilute to the volume with IPA and mix well. Vortex the solution followed by centrifugation at 12000 rpm for 3 minutes to aggregate the precipitate. The supernatant is then filtered through a 0.22 μm PTFE filter (discard the initial 2 mL) and transferred to an HPLC vial for injection.

### Example 2. HPLC Analytical Method: Cariprazine

A calibration curve was obtained through the HPLC method below to quantify the concentration of cariprazine in a sample with unknown API content.

### MATERIALS

### Reagents

Mill-Q water, resistivity greater than 18.0 MΩ-cm, or equivalent.
Ammonium acetate, ACS grade or equivalent
Sodium hydroxide, ACS grade or equivalent
Acetonitrile (ACN), HPLC grade

### Reference Standards

Cariprazine API with the defined potency

### Instruments and Parameters

### HPLC

Shimadzu HPLC System:
Binary Pump: Model LC-30AD
Degasser: Model DGU-20A
Autosampler: Model SIL-30AC
Column Oven: Model CTO-20AC
Detector: Model SPD-M30A

### Parameters

Column: Acquity UPLC BEH C18 Column, 130 Å, 1.7 μm, 3 mm×150 mm
Gradient:
Mobile Phase A: 0.05M ammonium acetate buffer, adjusted pH to 8 with 1N
NaOH/Acetonitrile=9/1
Mobile Phase B: ACN/Mill-Q water 9/1
Flow rate: 0.5 mL/min
Column temp: 30° C.
Injection vol: 5 μL
Detection: 248 nm
Run time: 15 min

Assay

| Time (min) | % MPA | % MPB |
|---|---|---|
| 0 | 45 | 55 |
| 1 | 45 | 55 |
| 4 | 40 | 60 |
| 6 | 38 | 62 |
| 9 | 10 | 90 |
| 10 | 10 | 90 |
| 15 | 45 | 55 |

### Example 3. GPC Analytical Method

Polymer MW was analyzed via gel permeation chromatography (GPC, also called size exclusion chromatography, SEC) as one key parameter for polymer selection on formulation development in this application.

### Materials

### Reagents

Tetrahydrofuran (THF), stabilized, HPLC grade.
N-methyl-2-pyrrolidone (NMP), pharma grade or ACS reagent.

### GPC Standards

GPC calibration kits: Pskitr1L ReadyCal-Kit (polystyrene), Mp: 266-66,000 Da are purchased from PSS-Polymer Standards Service USA Inc. Molecular weight information from the official document of ReadyCal-Kit, PSS-pskitril are listed in Table 1 below. Each vial of standards contains four polystyrene standards with different Mp.

### Instruments and Parameters

### GPC System

Shimadzu Nexera HPLC system:
Degasser: DGU-20A 5R
Binary pump: Model LC-30AD
RI detector: Model RID-10A
Autosampler: Model SIL-30AC
Column oven: Model CTO-20AC
Software: LabSolutions

### GPC Column

Two Agilent ResiPore (#1113-6300) 300×7.5 mm, 3 μm particle size columns in series.

### GPC Condition

Mobile Phase/Sample buffer: THF (stabilized).
Flow rate: 1 mL/min.
Column temp: 40° C.
Injection volume: 50 μL.
Run time: 30 minutes.
Reflective Index Detector:
Polarity: positive
Temperature: 40° C.
Response: 1.5 sec
Sample concentration: 2 mg (polymer)/mL in THF.

### Sample Preparation

### Standard Preparation

Prepare molecular weight standards (polystyrene) following the official instructions for PSS-pskitr1l ReadyCal-Kit. Add 1 mL of THF into each vial to make the standard solutions (3 individual STD vials to cover Mp 266-66000 Dalton) with concentration of 2.25 mg/mL for each standard. All standards are dissolved over 30 minutes. Note: Polystyrene standards and calibration curve shall be freshly prepared every time.

### Sample Preparation

For polymeric samples, weigh 10 mg of sample in a 1.5 mL Eppendorf tube. Add 1 mL of THF to dissolve polymer via an orbital shaker over 2 hr (room temperature). Centrifuge the dissolved polymer/THF sample at 14000 rpm for 2 minutes, take 100 μL of the supernatant for dilution to make the final 2 mg/ml sample for GPC analysis (100 μL of the supernatant+400 μL of THF).

For formulation samples, weigh a sufficient amount of formulation (corresponds to 10 mg of polymer) in a 1.5 mL Eppendorf tube. For example, for a 50% drug loading formulation with 50/50 polymer to biocompatible solvent ratio, 40 mg of the formulation shall be weighed. Centrifuge the dissolved formulation/THF sample at 14000 rpm for 2 minutes, take 100 μL of the supernatant for dilution to make the final 2 mg/ml sample for GPC analysis (100 μL of the supernatant+400 μL of THF).

### Example 4. In Vitro Release of VMAT2 Inhibitor From Injectable Suspensions

In vitro release was performed under sink condition for (+)-TBZ and (+)-(α)-HTBZ suspension formulations. Volume of the release medium could be adjusted according to the depot size and drug loading (%) of the formulation. In one embodiment, 35 mg of a 30% drug loading formulation was injected into 400 mL pH 7.4 phosphate buffer saline with 0.2% (v/v) Tween 80 at 37° C. After solvent dissipation, an in-situ forming implant would form in the release medium. At predetermined time points, 0.5 mL of the release medium was withdrawn for HPLC analysis to calculate drug concentration in the release medium. The accumulated amount of drug released was calculated at predetermined time points to obtain the accumulated release profile.

### Example 5. Polymer Molecular Weight Measurement

About 5-10 mg samples from each formulation was added into a 1.5 mL centrifuge tube and completely dissolved in 0.8 μL of THF. The solution was vortexed using a vortex shaker installed with a plate shaker until completely dissolved. Each sample was then centrifuged at 12,000 rpm for 2 minutes. The supernatant was collected and analyzed by GPC to determine the weight average molecular weight (Mw) and polydispersity index (PDI) of the polymer. Mw and PDI of a polymer were obtained by comparing with the polystyrene standards (Pskitr1L ReadyCal-Kit) with a Mp range from 266 to 66,000 Da.

### Example 6. Preparation of VMAT2 Inhibitor Containing Polymer Depot Compositions

(+)-TBZ, (+)-(α)-HTBZ, (+)-(b)-HTBZ, and valbenazine pharmaceutical compositions were prepared by filling weighed amount of API with desired particle size into a suitable luer-lock male syringe. A homogeneous polymer solution vehicle was prepared by mixing the weighed amount of polymer and biocompatible solvent(s) using a proper mixing device, e.g., a planetary mixer. Once prepared, a weighed amount of the polymer solution vehicle was filled into a suitable female, luer-lock syringe. Prior to injection, the male and female syringes were connected together, followed by back-and-forth mixing via the two plungers for up to 100 times to obtain uniform, milky, or slightly yellowish suspensions. More preferably, the mixing was 75 times, and still more preferably, the mixing was 50 times. The final mixture for injection can be a viscous liquid, a gel, an emulsion, a suspension, or a semisolid dispersion, which is stable and ready for injection for preferably 30 minutes and more preferably stable and ready for injection within 1-2 hours without sedimentation and aggregation. Once the suspensions were ready, the female syringe was detached and a desired luer-lock needle was screwed onto the male syringe for injection.

(+)-TBZ, (+)-(α)-HTBZ, (+)-(b)-HTBZ, and valbenazine pharmaceutical compositions can also be prepared as a prefilled single syringe ready for injection. Weighed amount of API with desired particle size were mixed with weighed amount of polymeric vehicle at predetermined ratio/drug loading via a pharmaceutical acceptable mixer to form the homogenous pharmaceutical compositions. The final pharmaceutical compositions can be a viscous liquid, a gel, an emulsion, a suspension, or a semisolid dispersion. The prepared pharmaceutical compositions are then filled into a pharmaceutically acceptable syringe which is then ready for injection. The pharmaceutically acceptable syringe can be composed of, but not limited to glass, polypropylene (PP), cyclic olefin copolymer (COC), and cyclic olefin polymer (COP). Preferably, a needle for injection was a 16-gauge needle, more preferably, an 18-gauge or 19-gauge needle, and most preferably, a 20-gauge or an even smaller sized needle.

### Example 7. Preparation of Sustained Release Pharmaceutical Compositions Composed of VMAT2 Inhibitor, a Poly(Lactide)-Polyethylene Glycol Diblock Copolymer, and Biocompatible Solvent(s)

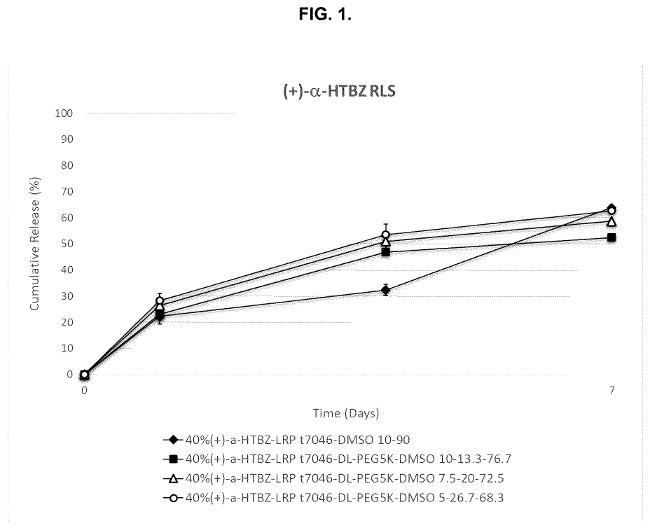

At 40% drug loading, a pharmaceutical composition composed of (+)-(α)-HTBZ, a diblock copolymer, [Poly(DL-lactide)-block-poly(ethylene glycol)methyl ether 5000; also known as DL-PEG 5K] and DMSO at 30-70 w/w ratio demonstrated sustained release for over 2 weeks with slightly less than 30% initial burst, followed by about 65% and about 70% in vitro release at Day 7 and Day 14, respectively (FIG. 1). It was also demonstrated that almost identical in vitro release profiles could be achieved when the weight ratio of the diblock copolymer DL-PEG 5K was kept in between 30-40% w/w ratio in the copolymer/DMSO vehicle solution (FIG. 1), which was advantages since the polymer ratio in a long acting injectable (LAI) depot formulation would significantly determine viscosity of the final drug product, thus, substantially affect its injectability. With less copolymer present in a pharmaceutical composition, a smaller injection force (less pain to a living subject being dosed) would be expected; or, in another approach, a smaller gauged needle could be adopted. In either way, patient compliance to a drug product could therefore be enhanced.

### Example 8. Preparation of Sustained Release Pharmaceutical Compositions Composed of VMAT2 Inhibitor, a Poly(Lactic-Co-Glycolic Acid)-Polyethylene Glycol Triblock Copolymer, and Biocompatible Solvent(s)

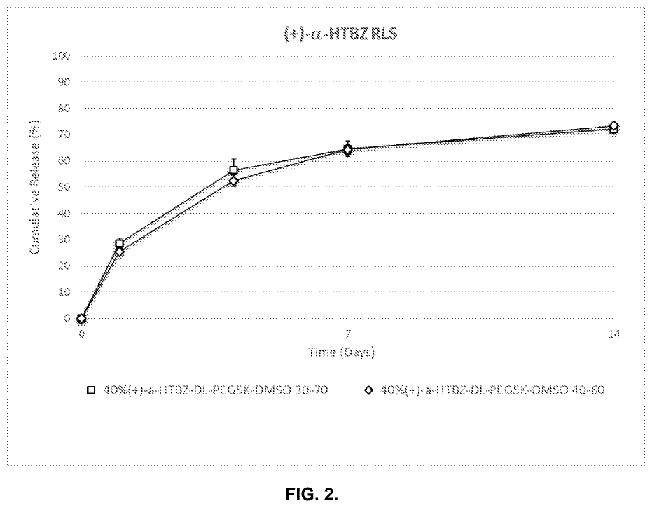

At 40% drug loading, a pharmaceutical composition composed of (+)-(α)-HTBZ, a triblock copolymer, [Poly(L-lactide-co-D,L-lactide-co-PEG); also known as LRP t7046] and DMSO at 10-90 w/w ratio demonstrated sustained release for over at least a week with about 20% initial burst, followed by about 30% and about 60% in vitro release at Day 4 and Day 7, respectively (FIG. 2).

It was also demonstrated that the detailed release profile within Day 1 to Day 7 could be fine-tuned by incorporation of poly(lactide)-PEG diblock copolymer [a combination of poly(lactide)-PEG diblock copolymer and poly(lactic-co-glycolic acid)-polyethylene glycol triblock copolymer]. For example, higher ratio of the poly(lactide)-PEG diblock copolymer in a pharmaceutical composition could accelerate the release rate of (+)-(α)-HTBZ, due to more hydrophilic nature of the poly (lactide)-PEG diblock copolymer, compare to more hydrophobic poly (lactic-co-glycolic acid)-polyethylene glycol triblock copolymer. Such finding could be advantageous. Sustained-release depot formulations made of poly (lactic-co-glycolic acid) (PLGA) are generally considered to last for more than several weeks. However, under certain circumstances, a shorter lasting sustained-release pharmaceutical composition may be desired (for example, last for 1 to 2 weeks), and the use of poly(lactide)-PEG diblock copolymer and poly(lactic-co-glycolic acid)-polyethylene glycol triblock copolymer in combination could be an excellent choice to tailor for appropriate release profile/duration.

### Example 9. Preparation of Antipsychotic Containing Polymer Depot Compositions

Cariprazine, lurasidone, brexpiprazole, aripiprazole, and paliperidone pharmaceutical compositions were prepared by filling weighed amount of API with desired particle size into a suitable luer-lock male syringe. A homogeneous polymer solution vehicle was prepared by mixing the weighed amount of polymer and biocompatible solvent(s) using a proper mixing device, e.g., a planetary mixer. Once prepared, a weighed amount of the polymer solution vehicle was filled into a suitable female, luer-lock syringe. Prior to injection, the male and female syringes were connected together, followed by back-and-forth mixing via the two plungers for up to 100 times to obtain uniform, milky, or slightly yellowish suspensions. More preferably, the mixing was 75 times, and still more preferably, the mixing was 50 times. The final mixture for injection can be a viscous liquid, a gel, an emulsion, a suspension, or a semisolid dispersion, which is stable and ready for injection for preferably 30 minutes and more preferably stable and ready for injection within 1-2 hours without sedimentation and aggregation. Once the suspensions were ready, the female syringe was detached and a desired luer-lock needle was screwed onto the male syringe for injection.

Cariprazine, lurasidone, brexpiprazole, aripiprazole, paliperidone pharmaceutical compositions can also be prepared as a prefilled single syringe ready for injection. Weighed amount of API with desired particle size were mixed with weighed amount of polymeric vehicle at predetermined ratio/drug loading via a pharmaceutical acceptable mixer to form the homogenous pharmaceutical compositions. The final pharmaceutical compositions can be a viscous liquid, a gel, an emulsion, a suspension, or a semisolid dispersion. The prepared pharmaceutical compositions are then filled into a pharmaceutically acceptable syringe which is then ready for injection. The pharmaceutically acceptable syringe can be composed of, but not limited to glass, polypropylene (PP), cyclic olefin copolymer (COC), and cyclic olefin polymer (COP). Preferably, a needle for injection was a 16-gauge needle, more preferably, an 18-gauge or 19-gauge needle, and most preferably, a 20-gauge or an even smaller sized needle.

### Example 10. Preparation of Lurasidone Containing a Poly(lactic-co-glycolic acid)-Polyethylene Glycol Triblock Copolymer, a mPEG-PLA Diblock Copolymer, and Biocompatible Solvent

A pharmaceutical composition composed of lurasidone, a triblock copolymer, (PLA-PEG-PLA) and a diblock copolymer (mPEG-PLA) in DMSO was made by first dissolving both the triblock and diblock copolymers in DMSO using a planetary mixer at a weight ratio of 20:80 polymer to solvent. The ratio of triblock to diblock copolymers was 1:6. Once a clear solution was obtained, lurasidone was added to the polymer solution at 50% by weight and mixed. The resulting formulation was filled into a 1 mL long syringe for injection.

### Example 11. Preparation of Cariprazine Containing a PLA-PEG-PLA Triblock Copolymer, a mPEG-PLA Diblock Copolymer, and Biocompatible Solvent

A pharmaceutical composition composed of cariprazine, a triblock copolymer, (PLA-PEG-PLA) and a diblock copolymer (mPEG-PLA) in DMSO was made by first dissolving both the triblock and diblock copolymers in DMSO using a planetary mixer at a weight ratio of 30:70 polymer to solvent. The ratio of triblock to diblock copolymers was 1:6. Once dissolved, cariprazine was added to the polymer solution at 20% by weight and mixed. The resulting formulation was filled into a 1 mL long syringe for injection.

### Example 12. Preparation of Brexpiprazole Containing a PLA-PEG-PLA Triblock Copolymer, a mPEG-PLA Diblock Copolymer, and Biocompatible Solvent(s)

A pharmaceutical composition composed of brexpiprazole, a triblock copolymer, (PLA-PEG-PLA) and a diblock copolymer (mPEG-PLA) in NMP was made by first dissolving both the triblock and diblock copolymers in NMP using a planetary mixer at a weight ratio of 20:80 polymer to solvent. The ratio of triblock to diblock copolymers was 1:6. Once dissolved, brexpiprazole was added to the polymer solution at 30% by weight and mixed. The resulting formulation was filled into a 1 mL long syringe for injection.

What is claimed is:

1. An injectable polymer depot composition comprising: i) a VMAT2 inhibitor or antipsychotic agent, a deuterated derivative thereof, a pharmaceutically acceptable salt thereof, a prodrug thereof; ii) one or more biodegradable, biocompatible block, branched, or dendritic copolymer or oligomer, or a mixture thereof; and iii) a pharmaceutically acceptable organic solvent selected from the group consisting of N-methyl-2-pyrrolidone, 2-pyrrolidone, ethanol, N,N-dimethylformamide, dimethyl sulfoxide, benzyl alcohol, benzyl benzoate, and combinations thereof.

2. The injectable polymer depot composition of claim 1, wherein the biodegradable polymer is a biodegradable diblock copolymer having the formula: methoxy poly(ethylene glycol)ᵥ-poly(lactic acid)_{w}, wherein v and w are the number of repeat units, wherein v is the number of repeat units ranging from 3 to 45 and w is the number of units ranging from 5 to 350.

3. The injectable polymer depot composition of claim 1, wherein the biodegradable polymer is a biodegradable triblock copolymer having the formula: poly(lactic acid)ₓ-poly(ethylene glycol)_{y}-poly(lactic acid)_{z} wherein x and z are the number of repeat units ranging from 20 to 700, wherein y is the number of repeat units ranging from 2 to 275 and x=z or x≠z.

4. The injectable polymer depot composition of claim 1, wherein the biodegradable polymer comprises (i) at least one biodegradable triblock copolymer having the formula: poly(lactic acid)ₓ-poly(ethylene glycol)_{y}-poly(lactic acid)_{z} wherein x and z are the number of repeat units ranging from 20 to 700 and y is the number of repeat units ranging from 2 to 275 and x=z or x≠z; and (ii) at least one biodegradable diblock copolymer having the formula: methoxy poly(ethylene glycol)ᵥ-poly(lactic acid)_{w}, wherein v and w are the number of repeat units, wherein v is the number of repeat units ranging from 3 to 45 and w is the number of units ranging from 5 to 350; and wherein the ratio of the biodegradable triblock copolymer of (i) and the biodegradable diblock copolymer of (ii) is 5:1 to 1:20.

5. The injectable polymer depot composition of claim 1, wherein the VMAT2 inhibitor is selected from the group consisting of (+)-TBZ, (+)-TBZ, (+)-(α)-HTBZ, (+)-(b)-HTBZ, deuterated (+)-TBZ, deuterated (+)-TBZ, deuterated (+)-(α)-HTBZ, and deuterated (+)-(b)-HTBZ.

6. The injectable polymer depot composition of claim 1, wherein the antipsychotic agent is selected from the group consisting of cariprazine, lurasidone, brexpiprazole, aripiprazole, paliperidone, lumateperone, asenapine, iloperidone, olanzapine, risperidone, quetiapine, ziprasidone, vortioxetine, vilazodone, duloxetine, mirtazapine, KarXT, a pharmaceutically acceptable salt thereof, a metabolite thereof, and a prodrug thereof.

7. The injectable polymer depot composition of claim 1, wherein the pharmaceutically acceptable organic solvent is selected from the group consisting of N-methyl-2-pyrrolidone and dimethyl sulfoxide (DMSO).

8. The injectable polymer depot composition of claim 1, wherein the amount of VMAT2 inhibitor or antipsychotic agent ranges from 5 to 80% by weight in the polymer depot composition.

9. The injectable polymer depot composition of claim 1 comprising 10% to 90% by weight of the biodegradable polymer.

10. The injectable polymer depot composition of claim 1, wherein the biodegradable block copolymer comprises at least one diblock copolymer of polylactic acid (polylactide) and polyethylene glycol (polyethylene oxide), at least one triblock copolymer of polylactic acid (polylactide) and polyethylene glycol (polyethylene oxide), and the combination thereof.

11. The injectable polymer depot composition of claim 1, wherein the biodegradable block copolymer comprises of at least one diblock copolymer of polylactic acid (polylactide), polyglycolic acid (polyglycolide), and polyethylene glycol (polyethylene oxide), at least one triblock copolymer of polylactic acid (polylactide), polyglycolic acid (polyglycolide) and polyethylene glycol (polyethylene oxide), and the combination thereof.

12. The injectable polymer depot composition of claim 1, wherein the biodegradable copolymer has an inherent viscosity between 0.20 to 0.80 dl/g.

13. The injectable polymer depot composition of claim 1, wherein the biodegradable copolymer has polyethylene glycol (polyethylene oxide) content in between 1 to 80% w/w.

14. The injectable polymer depot composition of claim 1 comprising 10% to 90% by weight of the pharmaceutically acceptable organic solvent based on the total amount of the biodegradable polymer and the pharmaceutically acceptable organic solvent.

15. The injectable polymer depot composition of claim 1, wherein the VMAT2 inhibitor or antipsychotic agent has a particle size distribution characterized by D(50) in a range from about 2 μm to about 300 μm.

16. The injectable polymer depot composition of claim 1, wherein the VAMT2 inhibitor or antipsychotic agent is uniformly dispersed in the composition and is filled in a syringe for subcutaneous or intramuscular injection.

17. A method of making the injectable polymer depot composition of claim 1, comprising: prefilling the antipsychotic agent or VAMT2 inhibitor, the deuterated derivative, the salt, the active metabolite, or the prodrug thereof in a syringe A; homogeneously mixing the biodegradable polymer and the pharmaceutically acceptable organic solvent to form a homogeneous mixture, and prefilling the homogeneous mixture in a syringe B; combining together syringes A and B, and thoroughly mixing components in syringe A and syringe B together to form the polymer depot composition.

18. A method of treating hyperkinetic movement disorder comprising administering a patient in need thereof the composition of claim 1 via injection; forming an in situ sustained release implant/depot upon administering to the patient; gradually releasing the VMAT2 inhibitor, the deuterated derivative thereof, the pharmaceutically acceptable salt thereof, the active metabolite thereof, or the prodrug thereof to the patient for at least one week.

19. A method of treating a psychiatric disorder, like schizophrenia, comprising administering a patient in need thereof the composition of claim 1 via injection; forming an in situ sustained release implant/depot upon administering to the patient; gradually releasing the antipsychotic agent, the deuterated derivative thereof, the pharmaceutically acceptable salt thereof, the active metabolite thereof, or the prodrug thereof to the patient for at least one week.

20. The method of claim 17, wherein release of the VMAT2 inhibitor or the antipsychotic agent, the deuterated derivative thereof, the pharmaceutically acceptable salt thereof, the active metabolite thereof, or the prodrug thereof from the in situ sustained release implant/depot is no more than 30% of the total amount of drug dosed 24 hours after the administration.
